(19) United States
(12) Patent Application Publication
(10) Pub. No.: US 2018/0049913 A1
(43) Pub. Date: Feb. 22, 2018

(54) POST-SURGERY THERMAL PACK HOLDING APPARATUS AND METHODS

(71) Applicants: Colette Spears, Charlotte, NC (US); Lenard Spears, Charlotte, NC (US)

(72) Inventors: Colette Spears, Charlotte, NC (US); Lenard Spears, Charlotte, NC (US)

(21) Appl. No.: 15/678,415

(22) Filed: Aug. 16, 2017

Related U.S. Application Data
(60) Provisional application No. 62/378,179, filed on Aug. 22, 2016.

(51) Int. Cl. A61F 7/02; A61F 7/10; A61F 7/08

(52) U.S. Cl. CPC A61F 7/02; A61F 2007/0001; A61F 7/08; A61F 7/10

(57) ABSTRACT

A thermal pack holding apparatus includes a thermal pack holder including an interior cavity configured to receive a thermal pack, the interior cavity bounded by a face panel and an opposed back panel, the face panel is thermally conductive and the back panel is moisture-resistant.

### CROSS-REFERENCE TO RELATED APPLICATIONS

This application claims the benefit of Provisional Application Ser. No. 62/378,179, filed Aug. 22, 2016.

### BACKGROUND OF THE INVENTION

This invention relates the applying of thermal therapy to help eliminate the pain and discomfort associated with post-surgical wounds.

“Cold”, ice, or “hot” packs (hereinafter referred to generically as “thermal packs”) are widely used to cool or heat body parts of the human anatomy as necessary to accelerate healing. Thermal packs are universally used to provide thermal therapeutic value to assist in the healing of post-surgical procedures. Thermal therapy reduces swelling and helps to minimize pain after surgery which ultimately reduces the number of painkillers a patient needs during recovery.

Cold in the form of ice or cold packs have been a time-honored and approved method to reduce the swelling and pain. One problem with the use of cold therapy post-surgery is the potential for introduction of moisture to the site of the surgical incision. This moisture increases the probability of a surgical incision becoming infected, thereby placing restrictions and limitations on the use of cold packs.

### BRIEF SUMMARY OF THE INVENTION

This problem is addressed by a thermal pack holder comprising a plurality of fabric layers configured to hold a thermal pack near a patient's skin while protecting the patient from moisture and abrasion.

According to one aspect of the technology described herein, a thermal pack holding apparatus includes a thermal pack holder including an interior cavity configured to receive a thermal pack, the interior cavity bounded by a face panel and an opposed back panel, the face panel is thermally conductive and the back panel is moisture-resistant.

According to another aspect of the technology described herein, a method for providing thermal therapy to a patient includes: placing a thermal pack at a predetermined temperature inside of an interior cavity of a thermal pack holder, wherein the interior cavity is bounded by a face panel and an opposed back panel, wherein the face panel is thermally conductive and the back panel is moisture-resistant; and applying the thermal pack holder against a patient's body, with the face panel in contact with the patient's skin.

### BRIEF DESCRIPTION OF THE DRAWINGS

The invention may be best understood by reference to the following description taken in conjunction with the accompanying drawing figures in which:

FIG. 1 is a schematic perspective view showing an exemplary thermal pack holder, along with a conventional thermal pack that may be placed within the holder;

FIG. 2 is a schematic, exploded perspective view showing the construction of the thermal pack holder, including an inner pocket and an outer pocket thereof;

FIG. 3 is a schematic diagram showing a portion of a thermal pack holder in contact with a patient's body part;

FIG. 4 is a schematic top plan view of a belt that may be used as part of a securing apparatus for a thermal pack holder;

FIG. 5 is a schematic top plan view of a shoulder strap assembly that may be used as part of a securing apparatus for a thermal pack holder;

FIG. 6 is a schematic front elevation diagram showing thermal packs secured in place against a patient's body using a securing apparatus; and

FIG. 7 is a schematic front perspective diagram showing another arrangement of thermal packs secured in place against a patient's body using a securing apparatus.

### DETAILED DESCRIPTION OF THE INVENTION

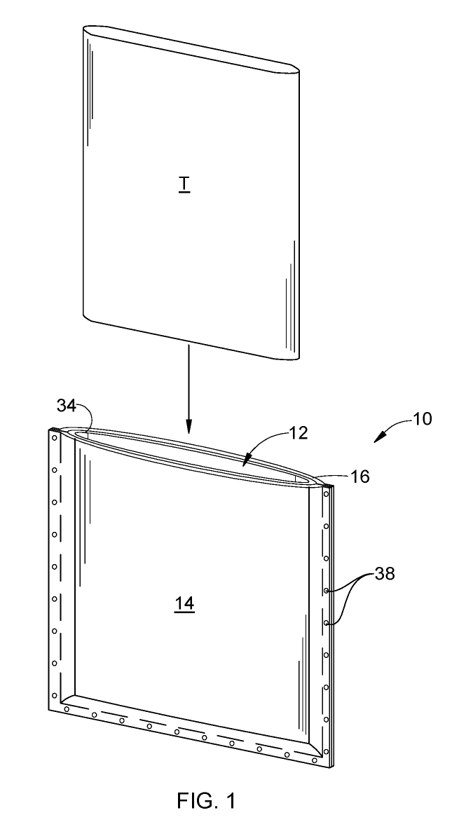

Referring to the drawings wherein identical reference numerals denote the same elements throughout the various views, FIG. 1 illustrates an exemplary thermal pack holder 10 constructed according to an aspect of the present invention. The thermal pack holder 10 is a flexible enclosure defining an interior cavity 12 which is configured (i.e. sized and shaped) to receive a conventional thermal pack, labeled “T”. As will be described in more detail below, the thermal pack holder 10 includes a face panel 14 configured for contact with a patient's skin, and an opposed back panel 16.

The thermal pack T may be any device which will fit into the thermal pack holder 10 which is effective to provide a thermal mass at a desired temperature (e.g. a “hot pack” or a “cold pack”). Numerous types of thermal packs are known and commercially available. For example, the thermal pack T may simply be a sealed container having a thermal mass (e.g. a solid or liquid material) which can be heated or cooled to a desired temperature. Alternatively, the thermal pack T may operate by combining two or more materials in an exothermic chemical reaction to produce heat. Alternatively, the thermal pack T may incorporate a material which may be frozen, such as water or water combined with a gelling material, which then thaws as it absorbs heat from a body part.

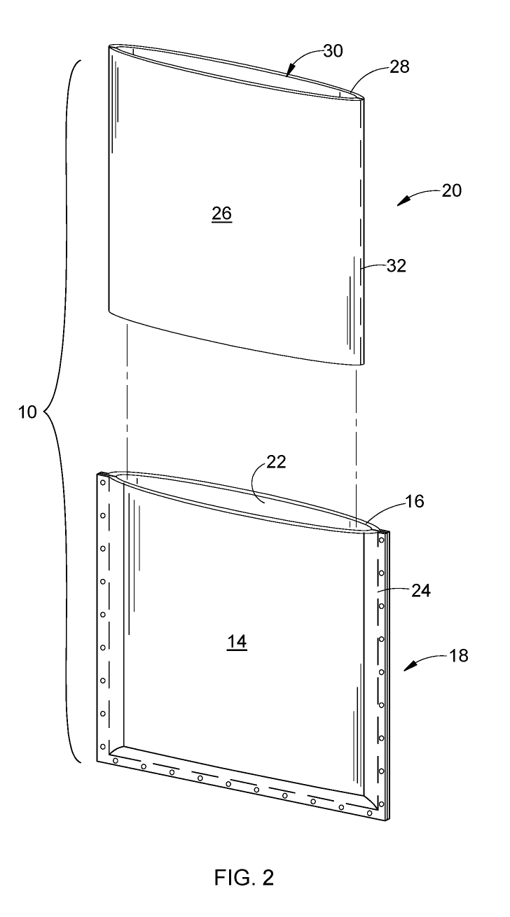

Referring to FIG. 2, the thermal pack holder 10 comprises an outer pocket 18 with an inner pocket 20 positioned therein.

The outer pocket 18 comprises the face panel 14 and the back panel 16. The panels 14, 16 are secured to each other along their respective peripheries in such a way as to define an opening 22. In the illustrated example, the outer pocket 18 is depicted as having a rectangular shape, but the specific shape is not critical, and other shapes such as a polygon, circle, oval, or a curvilinear shape could be substituted. As will be described in more detail below, the outer pocket 18 may be shaped and sized to conveniently fit against different human body parts as required.

The face panel 14 is intended to contact the patient's skin and comprises a material which readily permits thermal energy flow through its thickness. Stated another way, it is a thermally conductive material rather than an insulating material. While any thermally conductive material could be used, the material should be non-toxic. For example, a non-toxic synthetic material could be used. Preferably, the material of the face panel 14 is a natural material (e.g. cotton, wool, hemp, etc.) rather than a synthetic, so as to avoid any chance of including toxic compositions. The material the face panel 14 may be configured to be comfortable and avoid sticking to a patient's skin. One nonlimiting example of a suitable material is a cotton mesh fabric.

The back panel 16 is intended to contact the patient's clothing and may comprise a material which is moisture-proof in order to prevent the patient's clothing from getting wet. Optionally, the back panel 16 may be constructed from a material that is insulating, i.e. that tends to resist thermal energy flow through its thickness. The material of the back panel 16 may be a non-toxic synthetic material or a natural material (e.g. cotton, wool, hemp, etc.). One nonlimiting example of a suitable material is a knitted or woven wool fabric. If desired, a non-toxic chemical treatment or coating may be used with a natural material to provide moisture-proofing properties.

Any type of joint may be used between the face panel 14 and the back panel 16 so long as they are secured together, for example stitching, mechanical fasteners, folds, crimping, thermal bonding, or adhesives. In the illustrated example, conventional stitching 24 is used along three edges of the mutual perimeters of the panel 14 and the back panel 16, leaving the fourth edge open to define the opening 22.

The inner pocket 20 comprises a first panel 26 and a second panel 28. The panels 26, 28 are secured to each other along their respective peripheries in such a way as to define an opening 30. In the illustrated example, the inner pocket 20 is depicted as having a rectangular shape, but the specific shape is not critical, and other shapes such as a polygon, circle, oval, or a curvilinear shape could be substituted. As will be described in more detail below, the inner pocket 20 may be shaped and sized to match the shape of the outer pocket 18 as required.

The first and second panels 26, 28 are intended to directly contact the thermal pack T and comprise a material which readily permits thermal energy flow therethrough. The first panel 26 and the second panel 28 may comprise a material which is moisture-resistant in order to contain condensation generated by the thermal pack T. The material of the first panel 26 and the second panel 28 may be a non-toxic synthetic material or a natural material (e.g. cotton, wool, hemp, etc.). One nonlimiting example of a suitable material is a knitted or woven wool fabric. If desired, a non-toxic chemical treatment or coating may be used with a natural material to provide moisture-proofing properties.

Any type of joint may be used between the first panel 26 and the second panel 28 so long as they are secured together, for example stitching, mechanical fasteners, folds, crimping, thermal bonding, or adhesives. In the illustrated example, conventional stitching 32 is used along three edges of the mutual perimeters of the first panel 26 and the second panel 28, leaving the fourth edge open to define the opening 30.

The complete thermal pack holder 10 is formed by positioning the inner pocket 20 inside of the outer pocket 18. If desired, the inner pocket 20 and the outer pocket 18 may be joined to each other using one of the connection methods described above. The openings 22 and 30 coincide with each other and cooperatively define a mouth 34 (FIG. 1) that communicates with the interior cavity 12.

Optionally, a closure 36 (shown schematically) may be provided to selectively close off the mouth 34 of the thermal pack holder. Non-limiting examples of suitable closures include buttons, snaps, hooks, turn button fasteners, clips, hook and loop fasteners, or zippers.

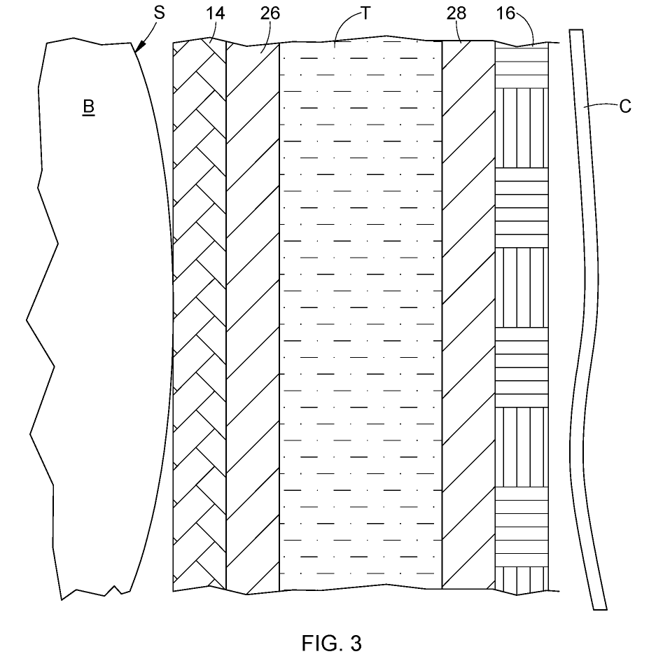

The thermal pack holder 10 is used (see FIG. 1) by placing a conventional thermal pack T into the inner pocket 20 and optionally securing the closure 36. The thermal pack holder 10 is then placed against a body part “B” (FIG. 3), with the face panel 14 in contact with the skin “S”. The face panel 14 is configured so that it does not cause achieving or irritation to the skin S. Optionally, clothing “C” may overlie the thermal pack holder 10.

As noted above, thermal packs T may be hot or cold. Especially in the case of a very cold or frozen cold pack, the thermal pack T may condense moisture out of the surrounding air. Because the inner pocket 20 is moisture-proof, it will retain any condensation and prevent it from touching the patient's body.

The thermal pack holder 10 may be placed against any body part as required. Typically, thermal pack therapy involves placing the thermal pack T against a body part and leaving it in place for a prescribed period of time. It is desirable that the patient be comfortable during this time and not have to hold the thermal packs T in place. Also, it is desirable that the patient be able to undertake normal activities during the thermal therapy.

Accordingly, the thermal pack holder 10 may be configured for use with a securing apparatus, by providing it with one or more connectors arranged in an appropriate pattern. Nonlimiting examples of suitable connectors include buttons, snaps, hooks, turn button fasteners, clips, hook and loop fasteners, or zippers. In the example illustrated in FIG. 1, the thermal pack holder 10 is provided with connectors 38 in the form of a plurality of spaced-apart snap fasteners arranged around its outer periphery. The snap fasteners or other connectors permit the thermal pack holder 10 to be connected to another thermal pack holder or to different kinds of securing apparatus.

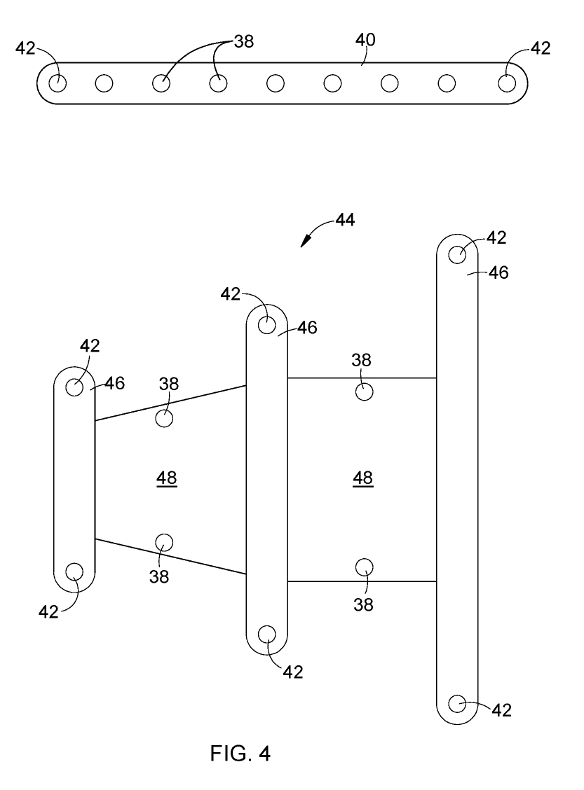

FIG. 4 illustrates an exemplary belt 40 constructed from an appropriate material such as leather, fabric, etc. and provided with end connectors 42. The belt 40 can be wrapped around a patient's torso or other body part and the end connectors 42 coupled to each other to secure the belt 40 in place. The belt 40 is provided with connectors 38 in the form of a plurality of spaced-apart snap fasteners arrayed along its length. The snap fasteners 38 or other connectors permit the belt 40 to be connected to a thermal pack holder 10 or to another piece of securing apparatus.

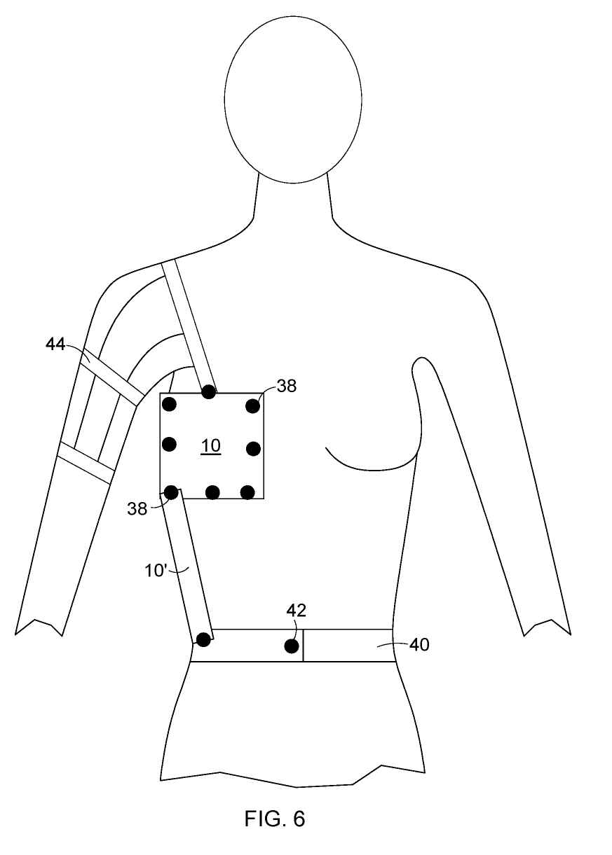

FIG. 5 illustrates an exemplary shoulder strap assembly 44 constructed from appropriate material such as leather, fabric, etc. The shoulder strap assembly 44 comprises two or more parallel spaced-apart straps 46 each having end connectors 42 at distal ends thereof, and a band of connecting material 48 interconnecting the straps 46. In addition to the end connectors 42, the shoulder strap assembly 44 may be provided with additional connectors 38 such as the illustrated snap fasteners. The end connectors 42 and/or the additional connectors 38 permit the shoulder strap assembly 44 to be connected to a thermal pack holder 10 or to another piece of securing apparatus.

The securing apparatus such as the belt 40 and the shoulder strap assembly 44, or other similarly-configured components may be used to secure one or more of the thermal pack holders 10 against one or multiple body parts for thermal therapy.

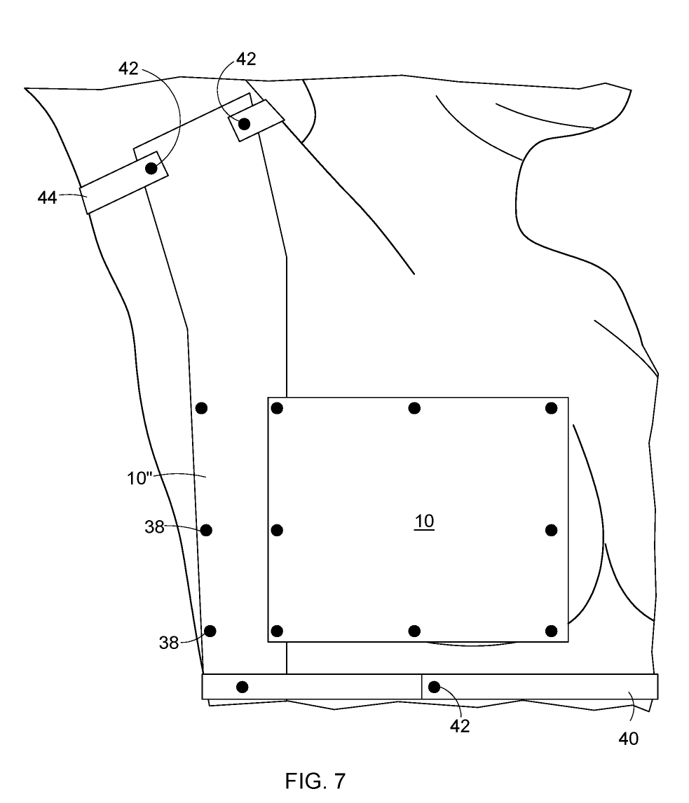

FIG. 6 illustrates one particular example in which thermal pack holders 10, 10′ are secured against a patient's breast and the side of the patient's thorax, respectively. In this example, the shoulder strap assembly 44 is placed over the patient's shoulder and the end connectors 42 are secured to the thermal pack holder 10, suspending it in position over the patient's breast. Another thermal pack holder 10′ contacting the patient's thorax is connected to the first thermal pack holder 10 at its upper end, and its lower end is secured in position by a belt 40 as described above.

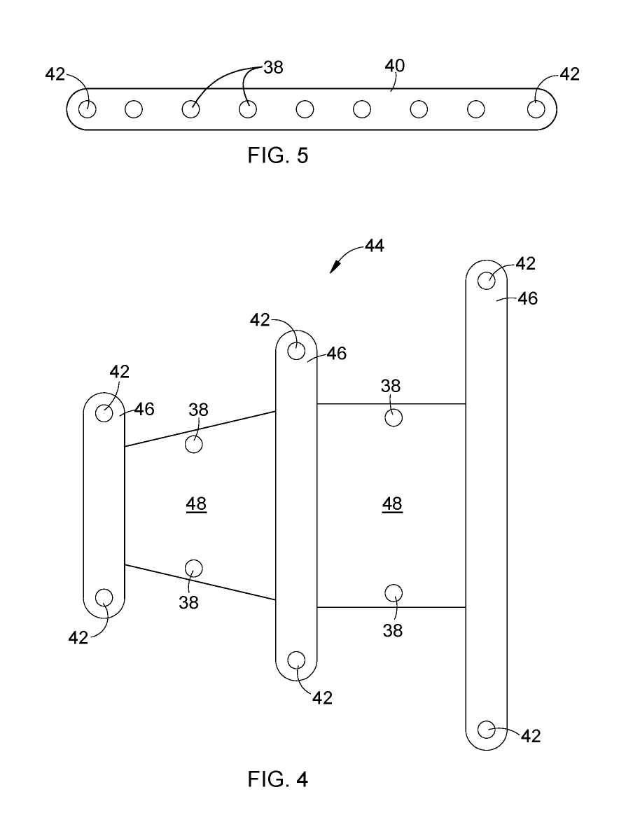

FIG. 7 illustrates another example in which a thermal pack holder 10″ is secured against a patient's upper thorax and armpit. This would be used, for example, when the patient has had a lymph node removal in conjunction with breast surgery. This configuration may be used separately from or in addition to the configuration shown in FIG. 6. In this example, the shoulder strap assembly 44 (or another strap) is placed over the patient's shoulder and the end connectors 42 are secured to the thermal pack holder 10″, suspending it in position against the patient's armpit. Its lower end is secured in position by a belt 40 as described above. Another thermal pack holder 10 may be connected to the first thermal pack holder 10″.

The thermal pack holder and securing apparatus described herein has advantages over prior art methods for thermal therapy.

It provides present an easy system and method for using thermal packs after surgical procedures while preventing the introduction of moisture into surgical incisions. It provides the individual who has undergone surgical procedures with a system of thermal packs are easily applied and can be customized to fit the necessary area affected by the surgical incision and surrounding traumatized body areas. The arrangement of connectors allows the thermal pack holders to be connected and customized to achieve the necessary coverage needed for the surgical incision and surrounding skin. It provides is designed to provide the appropriate pressure for the thermal pack to have contact with the surgical incision and skin in such a way that cooling or heating effects are maximized.

The foregoing has described an apparatus and method for post-surgical thermal therapy. All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

What is claimed is:

1. A thermal pack holding apparatus, comprising:
a thermal pack holder including an interior cavity configured to receive a thermal pack, the interior cavity bounded by a face panel and an opposed back panel, wherein the face panel is thermally conductive and the back panel is moisture-resistant.

2. The apparatus of claim 1 wherein the thermal pack holder comprises:
an outer pocket defined by the face panel and the opposed back panel; and
an inner pocket disposed inside the outer pocket and comprising a first panel and an opposed second panel, such that the inner pocket defines the interior cavity, wherein the first and second panels are moisture-proof.

3. The apparatus of claim 1 wherein the face panel comprises a mesh material.

4. The apparatus of claim 1 wherein the face panel and the back panel comprise natural fabrics.

5. The apparatus of claim 2 wherein the first and second panels comprise natural fabrics.

6. The apparatus of claim 1 wherein the face panel and the back panel are joined to each other along mutual peripheries thereof so as to define a mouth communicating with the interior cavity.

7. The apparatus of claim 1 further comprising a closure operable to selectively close the mouth.

8. The apparatus of claim 1 further comprising at least one connector attached to the thermal pack holder.

9. The apparatus of claim 1 further comprising a securing apparatus including at least one strap-like element having at least one connector configured for connection to the at least one connector of the thermal pack holder.

10. The apparatus of claim 9 wherein the securing apparatus includes a belt including a plurality of connectors configured for connection to the at least one connector of the thermal pack holder, and at least one end connector disposed at a distal end thereof.

11. The apparatus of claim 9 wherein the securing apparatus includes a shoulder strap assembly comprising two or more spaced-apart straps each having at least one and connector disposed at a distal end thereof, and a band of connecting material interconnecting the spaced-apart straps.

12. A method for providing thermal therapy to a patient, comprising:
placing a thermal pack at a predetermined temperature inside of an interior cavity of a thermal pack holder, wherein the interior cavity is bounded by a face panel and an opposed back panel, wherein the face panel is thermally conductive and the back panel is moisture-resistant; and
applying the thermal pack holder against a patient's body, with the face panel in contact with the patient's skin.

13. The method of claim 12 wherein the thermal pack holder comprises:
an outer pocket defined by the face panel and the opposed back panel; and
an inner pocket disposed inside the outer pocket and comprising a first panel and an opposed second panel, such that the inner pocket defines the interior cavity, wherein the first and second panels are moisture-proof.

14. The method of claim 12 wherein the face panel comprises a mesh material.

15. The method of claim 12 wherein the face panel and the back panel comprise natural fabrics.

16. The method of claim 13 wherein the first and second panels comprise natural fabrics.

17. The method of claim 1 further comprising using a securing apparatus including at least one strap-like element to secure the thermal pack holder against the patient's body.

18. The method of claim 18 wherein the thermal pack holder includes at least one connector, and the securing apparatus includes at least one connector configured for connection to the at least one connector of the thermal pack holder.

19. The method of claim 18 wherein the securing apparatus includes a belt including a plurality of connectors configured for connection to the at least one connector of the thermal pack holder, and at least one end connector disposed at a distal end thereof.

20. The method of claim 18 wherein the securing apparatus includes a shoulder strap assembly comprising two or more spaced-apart straps each having at least one and connector disposed at a distal end thereof, and a band of connecting material interconnecting the spaced-apart straps.
